SEQUENCE LISTING (4 sequences)

SEQ ID NO: 1 - DNA, 20 bp, artificial
  primer Btub1
atgcaagaaagccttgcgac

SEQ ID NO: 2 - DNA, 20 bp, artificial
  primer Btub2
tttgcagtatctcagtgttc

SEQ ID NO: 3 - DNA, 22 bp, artificial
  primer R1CIRE
cacttaggggtcctcgaatgta

SEQ ID NO: 4 - DNA, 22 bp, artificial
  primer ERIC2
aagtaagtgactggggtgagcg

(19) United States
(12) Patent Application Publication
(10) Pub. No.: US 2009/0169518 A1
(43) Pub. Date: Jul. 2, 2009

(54) Pochonia chlamydosporia strain pcmr and method to use it in biological control of the root-knot-nematode (meloidogyne spp)

(75) Inventor: Carlos Manuel Antunes Franco, Évora (PT)

Correspondence Address: SUGHRUE MION, PLLC, 2100 PENNSYLVANIA AVENUE, N.W., SUITE 800, WASHINGTON, DC, 20037, US

(73) Assignee: universidade de evora, Evora (PT)

(21) Appl. No.: 11/629,124

(22) PCT Filed: Jun. 9, 2005
(86) PCT No.: PCT/IB2005/051899

§ 371 Date: Nov. 19, 2007

(30) Foreign Application Priority Data
Jun. 11, 2004 (PT) 103146

(51) Int. Cl. A01N 63/04; C12N 1/14; A01P 5/00

(52) U.S. Cl. 424/93.5; 435/254.1

(57) ABSTRACT

This invention refers to isolation and utilization of the fungus Pochonia chlamydosporia var. chlamydosporia strain PcMR in biological control of nematodes, characterized in that it has a high virulence against Meloidogyne spp. in in-vitro microbiologic tests, pot tests and field tests. In these tests, the strain PcMR, isolated in Portugal, have a good performance in the ability to control Meloidogyne spp. populations and shows a good capacity to produce high amounts of chlamydospores used to produce inoculum and to colonize plant roots that might be infected by Meloidogyne spp. This strain can be utilized as part of biological control methods that effectively control root-knot-nematode populations belonging to genus Meloidogyne and this invention refers also to production and utilization of nematicides based on Pochonia chlamydosporia var. chlamydosporia strain PcMR and any organisms derived from this strain.

### TECHNICAL FIELD OF INVENTION

This invention relates to control methods to use in Plant health management programs. Provides an active ingredient and a method of biological control against diseases caused by the root-knot-nematodes that belong to the genera Meloidogyne spp. In this particular form provides a strain of the fungus Pochonia chlamydosporia and a method of using it as a way of decrease or inhibit Meloidogyne spp. populations developing in soil and rhizosphere of susceptive plants.

### BACKGROUND ART

The root-knot nematodes, like other plant pathogenic organisms can be controlled using several control methods, namely phisic, cultural, genetic, chemical and biological, and/or a combination of these, following different strategies, namely the Integrated Pest Management which arouse as the main strategy over the latest years. This strategy aims to decrease the undesirable side-effects of Pesticide use, and the optimisation of these and other control methods, ensuring at the same time the objectives of the plant growers and the minimization to the lowest possible level of the secondary effects on the environment and consumers.

Although the cultural and physical control methods currently available, may achieve in particular conditions a certain degree of success, they are not suited for the most practical situations where the most important economic damages arouse from the presence of significant populations of Meloidogyne spp., because they require high levels of resources, namely economic and time.

With genetic methods, a partial success has been achieved by plant breeding, but the strains currently available and economically more interesting for plant growers, can't resist or have low tolerance to Meloidogyne spp. attack. Nevertheless, can be observed great tolerance variability among plants and strains, and is expected that those with higher levels of tolerance are in better position to succeed in commercial growers.

Chemical control methods are the main process used to limit the Meloidogyne spp. populations, especially in high value crops that use high amounts of input factors.

There are 3 main pesticide groups that are in use to control nematodes: general fumigant biocides; fumigant nematicides; non-fumigant nematicides.

The general biocides are the most effective, probably due to its physic properties that allow its even distribution in the soil, which is mainly due to the high vapour pressures at room temperature. One example of these compounds is the most widespread named metal-bromide. These compounds have several drawbacks: they have to be applied before crop settlement, because they are phytotoxic and demand a waiting period variable with the substance, usually between 1-4 weeks, and cause noxious side-effects on the environment.

The fumigant nematicides, although effective under the appropriate conditions, present more variability in the efficacy, because they experience a higher degree of difficulty to spread in soil. They present also similar drawbacks to the previous group.

The non-fumigant nematicides, although effective in favourable conditions, are usually less effective compared to the compounds of the previous groups, specially due the difficulty in spreading them evenly through the soil, although they can be used after crop settlement, which is an advantage, because they are less phytotoxic.

All the referred pesticides have several undesirable side-effects, and although these vary with the substance, all have side-effects that can be considered a liability for public human health, and for the environment, if their use becomes widespread.

In summary, for the major substances currently in use:

General biocides: metal bromide goes to atmosphere and is a strong depletor of ozone layer, and its use is currently prohibited by the international environment agreements. The chlorpicrin and metyl-isothiocianate precursors (dazomet and metham-sodium) left dangerous residues in crops for several months and can contaminate underground water.

Fumigant nematicides: The 1.3 D, the only allowed currently in most countries, left residues in soil for several months, can contaminate underground water, left residues in plants and is highly phytotoxic.

Non-fumigant nematicides: All the 4 more widespread, left high levels of residues in crops, during large periods of time, which limit its homologation to long cycle crops and with long safety intervals which limits its efficacy. Because many of them act by inhibiting nematode actions and not by killing them, they can recover after the soil concentration lower to adequate levels, as happen with fenamifos and oxamil. Besides those, the substances carbofuran and aldicarb are highly dangerous for underground water; and their use is limited to situations where the risk of underground contamination is low.

Biological control methods are an alternative to the methods presented above, but in spite of the strong pressure coming from the side effects of pesticides and lack of other viable alternatives, currently there are very few effective alternatives that can be used to control Meloidogyne spp. populations.

These nematodes possess many natural enemies that can be searched for new methods of biological control. They belong to many different groups of organisms, like bacteria, insects, acariens, predator nematodes and fungi. Although many of these organisms have been studied, there are 4 that received the major efforts from the researchers over the years, although its effective use as part of a control method has proved to be difficult. These organisms are: the bacteria Pasteuria penetrans, and 3 types of fungus: species of the genus Artrobotrys spp. namely A. Dactyloides and A. Oligospora, the fungus Paecilomyces lilacinus and the fungus Pochonia chlamydosporia, named previously Diheterospora chlamydosporia or Verticillium chlamydosporium.

Currently to any of these organisms, its possible identify two different strategies of control against Meloidogyne spp. populations.

a) Promote the development of indigenous populations

b) Introduce relatively small inoculations, or mass inoculations, with strains exogenous to the ecosystem, in order to enable the settlement of an aggressive population of nematode antagonists, capable of control the nematode population.

To the present date it was not possible, with pontual exceptions in particular environments, not fully understanded and concerning both strategies, to produce a reliable and reproducible biological method of control against Meloidogyne spp.

Concerning the fungus Pochonia chlamydosporia there are extensive published data (Kerry, 1995; Kerry & Bourne 1996; Kerry & Bourne, 2002; Bourne, et al., 1996; Leij et al., 1992; Leij et al. 1993), suggesting the possibility of using strains of this fungus as part of control strategies against Meloidogyne spp. mainly by the use of great amounts of spores, by mass inoculate them in the treated soils.

To use the referred strategy its necessary that the selected strain meet the following requirements: i) possess a high agressivity against Meloidogyne spp. eggs, which is measured by the % parasitism, measured in Petri dishes, and should be higher than 30%; ii) high ability to colonize the roots, which is measure in Petri dishes and should be bigger than 80%; iii) high capacity to produce chlamydospores in solid media, using milled barley, and should be bigger than 10⁶ chlamydospores/g media in order to permit enough spore production to built the inoculum for field inoculation.

In addition, in pot tests, it was possible to determine the main variables related to parasitism, namely: i) the population recovered from soil, should be higher than 2×10⁴ cfu/g soil (colony forming units); ii) the rhizosphere population should be bigger than 8×10³ cfu/g root; and iii) the egg parasitism, extracted from infected roots with Meloidogyne spp. should be higher than 30% (parasitised eggs/total number of eggs).

In spite of the results obtained in controlled conditions in laboratory tests, in pot tests and in microplots, it was not possible to confirm them in commercial crops, growing in the conditions similar to those of commercial growers, either using indigenous populations or with mass introduction of formulated spores of exogenous strains, grown specially to that purpose.

In spite of that, and based only in laboratory tests and in pots there is a strain of Pochonia chlamydosporia named at the time, Verticillium chlamydosporia strain ac, whose use against Meloidogyne spp. is patented (WO91/01642).

### SOLUTIONS PROVIDED BY THIS INVENTION

The majority of the strains discovered to date, are not good candidates to use in biological control, because they have a relative low virulence against Meloidogyne spp. eggs. This is shown by in-vitro tests, yielding a parasitism rate average that seldom surpass 40%. Besides that, in field conditions similar to commercial crops, and using as inoculum chlamydospores formulated with inert materials, it was not possible to date obtain significant population reductions with any of the tested strains.

The strain PcMR has a high virulence against Meloidogyne spp. eggs, and shows a parasitism rate of 66% in in-vitro Petri dish selection tests.

The strain PcMR, in conditions similar to those utilised by commercial growers, can control Meloidogyne spp. populations in root-knot-nematode infested soils. This control can be achieved at least in two consecutive years, in soils inoculated with chlamydospores formulated with sterilized fine sand (Ø 90-700 μm), with a rate of 5000 chlam./g soil, in the most superficial 20 cm of soil and can be shown by the following parameters:

a) Meloidogyne population reductions to control of 74% in the total number of viable juveniles plus eggs (reduction between 56 and 91%, t-student 95% interval) in 1^{st} year, and 65% (reduction between 60 and 71%, t-student 95% interval) in the 2^{nd} year.

b) Meloidogyne population reductions to control of 64% in the total number of viable juveniles+eggs (reduction between 49 and 79%, t-student 95% interval) in 1^{st} year, and 70% (reduction between 63 and 77%, t-student 95% interval) in the 2^{nd} year.

c) An average PcMR egg parasitism (eggs collected from egg masses present in the outside of roots) of 45% (between 39 and 51%, t-student 95% interval) in 1^{st} year, and of 39% (between 34 and 43%, t-student 95% interval) in the 2^{nd} year.

The strain PcMR is characterized in that it has all the other characteristics, consider essential to be utilized as biologic control agent, referred in background art, and characterized in the detailed description.

### DETAILED DESCRIPTION

Morphologic Characterization of PcMR Strain:

This strain of the fungus Pochonia chlamydosporia has all the morphologic typical characteristics of the fungus Pochonia chlamydosporia var. chlamydosporia, described by Gams, 1988.

Selection tests outcome, using the methods stated in IOBC manual (Kerry & Bourne, 2002):

Were utilized the 3 standard selection tests, the Barley root colonization test in semi-sterile conditions, the chlamydospore production test in vitro, the egg parasitism test in water-agar Petri dishes.

PcMR yielded the following results, in these in-vitro selection tests:

a) Barley roots colonization test: 81.4%.

b) Chlamydospore production test: 2.2×10⁷ chlamydospores/g of barley used in the chlamydospore production medium.

c) Egg parasitism test: 66%.

Green pepper pot tests according to IOBC methods (Kerry & Bourne, 2002):

Experiment Conditions:

The experiment was carry way in a growth chamber, at 25° C., with appropriate light regime to green pepper growth, and with manual irrigation.

Plant utilized: Green Pepper, Capsicum annum var. Corteso.

Soils used: 2 types: type 1: non-autoclaved sandy soil; type 2: autoclaved sandy soil.

Fungus treatment: 3 strains tested and controlled (non-inoculated soil). All inoculations were performed with 5000 chlamydospores/g of soil.

Nematode treatment with Meloidogyne incognita: were used 3 levels: level 1: 6000 juveniles, level 2: 2500 juveniles, level 3: control (non-inoculated).

Each statistical unit, obtained by the combination of the different treatments stated was repeated 5 times.

In pot tests the strain PcMR decreased significantly (S<0.05) the number of eggs/egg mass, in soil inoculated with 5000 chlamydospores/g of soil, compared to non-inoculated soil, in eggs collected from egg masses obtained from the green peppers grown in the pots with 2500 and 6000 juveniles of Meloidogyne incognita.

Field Trials, in 2 consecutive years, in conditions matching those utilized by commercial growers (assay design and evaluation methods of assay parameters according to Kerry & Bourne, 2002)

Assay Description:

Site: Agricultural Experimental station of Patação, in Direcção Regional de Agricultura do Algarve, Algarve, Portugal.

Plastic house structure: Plastic house length-40 m, width-7.2 m.

The plastic house soil was a Meloidogyne javanica infested sandy soil, and had no indigenous population of P. chlamydosporia.

Cultivated plant: Tomato, Lycopersicum esculentum var. sinatra.

Plant density: 6 rows with 97 plants each, spaced 1.1 m between rows×0.4 m in the row.

Statistical Design:

16 plots, each with 3 plant rows, 11 plants per row, in a total of 33 plants per plot. The plots were randomly distributed in the plastic house.

Fungus treatment: only one strain tested: strain PcMR

Were chosen 3 treatments and 1 control (without treatment).

Treatment 1: nematicide application: methyl-bromide in pre-plantation.

Treatment 2: nematicide application+PcMR: methyl-bromide in pre-plantation+PcMR treatment (same as treatment 3).

Treatment 3: PcMR treatment: 2-3 inoculations, utilizing 5000 chlamydospores/g soil, inoculating 8 plants chosen in central row of the plot, and calculating the soil volume by multiplying soil area available for each plant×top 20 cm of soil. The inoculations started 2-3 weeks after planting.

Treatment 4: control: no treatment applied.

Each treatment was repeated 4 times. All the evaluated parameters were determined 3 times in each sample.

The Results:

1^{st} year (Treatment 3 compared with control): Soil colonization, final population recovered at the end of assay: 2.7×10⁴ cfu/g of soil (conf. int. t-student 95%, between 1.5 and 3.8×10⁴ cfu/g of soil).

Root colonization: 1.3×10⁴ cfu/g of root (conf. int. t-student 95%, between 0.7 e 1.9×10⁴ cfu/g of root)

Egg parasitism in Meloidogyne eggs, tested in semi-selective media, from eggs extracted by mechanic methods from egg masses obtained from the field infected plants: average 45% (conf. int. t-student 95%, between 39 and 51%).

Meloidogyne population reduction: Determination of the parameter: total viable juveniles plus eggs: average of 74% (conf. int. t-student 95%, between 56 e 91%). Determination of parameter: number of viable eggs/egg mass: average of 64% (conf. int. t-student 95%, between 49 e 79%).

2^{nd} year (Treatment 3 compared with control): Soil colonization, final population recovered at the end of assay: 2.8×10⁴ cfu/g of soil (conf. int. t-student 95%, between 1.7 and 3.8×10⁴ cfu/g of soil).

Root colonization: 1.3×10⁴ cfu/g of root (conf. int. t-student 95%, between 1.2 e 1.5×10⁴ cfu/g of root).

Egg parasitism in Meloidogyne eggs, tested in semi-selective media, from eggs extracted by mechanic methods from egg masses obtained from the field infected plants: average 39% (conf. int. t-student 95%, between 34 and 43%).

Meloidogyne population reduction: Determination of the parameter: total viable juveniles plus eggs: average of 65% (conf. int. t-student 95%, between 60 e 71%). Determination of parameter: number of viable eggs/egg mass: average of 70% (conf. int. t-student 95%, between 63 e 77%).

### INDUSTRIAL APPLICATIONS

A—This strain can be utilized in mass production of chlamydospores, using fermentors of variable size, using the appropriate technology to produce large amounts of chlamydospores, namely solid state fermentors using an appropriate substratum. Currently, several media can be used, namely media based in barley, rice, wheat or corn. These spores can then be extracted from liquid suspensions (international patent request PTI 04/000009), or through dry methods.

B—Its possible to obtain formulated products from extracted chlamydospores referred in A by adding to them inert substances. These products can be applied in soils, in plants, in seeds or other growing media for plants. These products can be utilized as part of a biological control method against root-knot-nematode (Meloidogyne spp.) which can be utilized in the majority of susceptible cultivated plants.

### EXAMPLE

Control of a Meloidogyne spp. population, composed mainly by Meloidogyne javanica, in plastic house tomato crop, in Algarve, Portugal.

Mass Production of Chlamydospores.

1—Production medium preparation (barley+sand medium) (Kerry & Bourne 2002):

The barley is milled and sieved through a 2 mm sieve and then washed through a 44 μm, collecting all the residue collected in 44 μm.

Sieve fine sand, through a 2 mm sieve, then wash it through a 44 μm, and then collect the particles between 2 mm and 44 μm.

The production medium is obtained by mixing the collected fractions of milled barley and fine sand in 1:1 proportion, and let it dry to the appropriate moisture level.

Place 50 ml of this mixture in small 250 ml Erlenmyer flasks (fermentors), close with cotton, seal with aluminium foil and then autoclave.

2—Inoculate each fermentor of 250 ml, in sterile conditions, with 4 agar plugs previously colonized with PcMR, wait 3 days at 25° C., and then shake gently to spread inoculum and then let incubate for 1 month.

Chlamydospore Extraction.

From a sufficient number of 250 ml fermentors, extract all the colonized medium with chlamydospores, homogenise and wash it to the feeding tank of the extraction and separation apparatus. Then operate the apparatus in order to correctly extract the chlamydospores (international patent request PTI 04/000009) and then collect the chlamydospores in appropriate vials, and keep them in the fridge at 4° C.

Product Formulation:

Prepare fine sand (Ø 90 to 700 mm), sterilize it in the autoclave, dry it overnight at 90° C. in the oven.

Build up the inoculum by mixing the sand and chlamydospores, evenly at the 10:1 proportion, and then keep it at 4° C. until necessary.

Plant Inoculation:

With soil moisture at field capacity, apply an aqueous mixture of inoculum to the plant by spreading it on the soil around the plant, at the rate of 5000 chlamydospores/g soil, on the top 20 cm of the soil. Then slightly incorporate inoculum in soil surface and cover it with soil.

Apply the inoculum as much times as needed to achieve a recovered population from soil of 2×10⁴ cfu/g soil.

Soil Monitorization:

Monitoring Pochonia chlamydosporia var. chlamydosporia strain PcMR. Perform soil analysis, with the available methods, to determine the population of PcMR in cfu/g soil. (IOBC manual, Kerry & Bourne, 2002).

### BIBLIOGRAPHY

Gams, W. (1988) A contribution to the knowledge of nematophagous species of Verticillium. Netherlands Journal of Plant Pathology, 94: 123-148.
Kerry, B. R. and Bourne, J. M. (ed.) (2002) A Manual for Research on Ver-ticillium chlamydosporium, a Potential Biological Control Agent for Root-Knot Nematodes. IOBC/WPRS, Gent, Belgium, 84 pp.
Kerry, B. R. (1995) Ecological considerations for the use of the nematophagous fungus, Verticillium chlamydosporium, to control plant parasitic nematodes. Canadian Journal of Botany 73(suppl. 1): S65-S70.
Kerry, B. R. and Bourne, J. M. (1996) The importance of rhizosphere interactions in the biological control of plant parasitic nematodes-a case study using Verticillium chlamydosporium. Pesticide Science 47: 69-75.
Bourne J. M., Kerry, B. R., and Leij, F. A. A. M. de (1996) The importance of the host plant on the interaction between root-knot nematodes (Meloidogyne spp.) and the nematophagous fungus, Verticillium chlamydosporium Goddard. Biocontrol Science and Technology 6 (4), 539-548.
Leij, F. A. A. M. de, Davies, K. G. and Kerry, B. R. (1992) The use of Ver-ticillium chlamydosporium Goddard and Pasteuria penetrans (Thorne) Sayre & Starr alone and in combination to control Meloidogyne incognita on tomato plants. Fundamental and Applied Nematology 15(3), 235-242.
Leij, F. A. A. M. de, Kerry, B. R. and Dennehy, J. A. (1993) Verticillium chlamy-dosporium as a biological control agent for Meloidogyne incognita and M. hapla in pot and micro-plot tests. Nematologica, 39: 115-126.

1. Fungus Pochonia chlamydosporia var. chlamydosporia, strain MR, isolated in Portugal, with strong parasitism activity over animals of phylum nematoda characterized in that, the parasitism tests in vitro, originate a parasitism rate of 66% in Meloidogyne spp. eggs, in pot tests gave a significant reduction in the number of eggs per egg mass of Meloidogyne incognita in green pepper crop, and in field tests, on tomato crops in plastic house with similar conditions to those used by growers for commercial purposes, make a reduction of 68% in the total number juveniles+eggs, of 70% in the number of viable eggs per eggs mass and 42% of parasitism on the collected eggs from egg masses.

2. Fungus according to the claim 1, that in microbiology tests in vitro is characterized in that it colonizes 81.4% of barley roots and produce 2.2×10⁷ chlamydospores per gram of barley used in the solid medium on its production.

3. Fungus according to the claim 1, that obtain a single DNA fragment with 519 bp on B-tubulin PCR using the primers Btub1 (SEQ ID NO: 1) and Btub2 (SEQ ID NO: 2), characterized in that it obtain on Eric PCR, using the primers, R1CIRE (SEQ ID NO: 3) and ERIC2 (SEQ ID NO: 4) and ignoring all the fragments below 100 bp, 5 fragments with the following sizes, 1^{st} with 100 bp, 2^{nd} with 200 bp, 3^{rd} with 250 bp, 4^{th} with 400 bp and 5^{th} with 500 bp.

4. A nematocidal composition characterized in that its active ingredient is based on the fungus Pochonia chlamydosporia variety chlamydosporia, strain MR, according to what was described in claim 1, and to be constituted by aleurospores type conidia (also designated by dictyochlamydospores or simply chlamydospores), in formulation with inert material or other type of organic or inorganic adjuvant.

5. Nematocidal composition according to claim 4 characterised in that it is used in the control of nematode populations of genus Meloidogyne.

6. Nematocidal composition according to claim 5 characterised in that it is used in the control of Meloidogyne incognita and Meloidogyne javanica.

7. Use of a nematocidal composition according to claim 4 characterised in that it is distributed through solid spreading or through an aqueous mixture.

8. Method to control Meloidogyne species, that use the fungus Pochonia chlamydosporia variety chlamydosporia strain MR, or any variant or mutant obtained from this strain, according to claim 1 characterised in that it can be applied to plant seeds, plant roots, soil, or any other plant growing substratum, or in any other situation aiming the control of Meloidogyne spp. populations.
